(19) United States
(12) Patent Application Publication
(10) Pub. No.: US 2018/0055957 A1
(43) Pub. Date: Mar. 1, 2018

(54) Disinfection Box

(71) Applicants: Li-Chou Lu, New Taipei City (TW); Shin-Nan Hsieh, New Taipei City (TW); Jung-Yen Liao, New Taipei City (TW); Wei-Hsien Lai, New Taipei City (TW); Ching-Hai Yao, New Taipei City (TW)

(72) Inventors: Li-Chou Lu, New Taipei City (TW); Shin-Nan Hsieh, New Taipei City (TW); Jung-Yen Liao, New Taipei City (TW); Wei-Hsien Lai, New Taipei City (TW); Ching-Hai Yao, New Taipei City (TW)

(21) Appl. No.: 15/684,816

(22) Filed: Aug. 23, 2017

(30) Foreign Application Priority Data
Aug. 24, 2016 (TW) 105212855

(51) Int. Cl. A61L 2/07; A61L 2/10; A61L 2/18

(52) U.S. Cl. CPC A61L 2/07; A61L 2/20; A61L 2/18; A61L 2/10

(57) ABSTRACT

A disinfection box includes a disinfection box unit, ultraviolet disinfection lamps, air dehumidification equipment and ozone generation equipment. The ozone generation equipment is used along with the ultraviolet disinfection lamps to sterilize and disinfect medical equipment to be disinfected inside the disinfection box unit. In particular, as the equipment box is rotatable, the medical equipment to be disinfected can be fully disinfected without any blind spot. In addition, by the collaborative use of the ultraviolet disinfection lamps, the air dehumidification equipment and the ozone generated by the ozone generation equipment, the medical equipment to be disinfected can be sterilized and disinfected quickly and sufficiently in a short time, thereby saving time as well as being sterilized and disinfected without any blind spot.

### FIELD OF THE INVENTION

The present invention relates to a disinfection box, and more particularly to a disinfection box used as medical equipment.

### BACKGROUND

For an ordinary place with medical procedures, such as an operating room, a dental clinic or a general clinic where patients should be touched with medical devices, the medical devices have to be used in treating or examining the patients; whereas, the used medical devices must go through sterilization and disinfection in high-pressure steam. However, for an existing disinfection box using high-pressure steam, a lot of electricity is required to maintain the high-pressure operation, which consumes energy considerably. In addition, as steam must be generated through high pressure, there exists a certain high risk for high-pressure equipment. On the other hand, as steam is used in the disinfection process, a disinfection chamber will be always under a moist condition during a long time of use, and invisible fungi or bacteria will be definitely generated. More importantly, the medical equipment that require disinfection is mostly put flat, therefore, the effective and complete disinfection cannot be performed to the area where the medical equipment are overlapped. As the disinfection with high-pressure steam must maintain for a long time to achieve the effective sterilization and disinfection, the institutes using a large quantity of medical devices must prepare plural sets of medical devices, which is rather inconvenient and costly.

### SUMMARY OF THE INVENTION

Accordingly, the primary object of the present invention is to provide a disinfection box. As ozone is a kind of gas with a high efficient disinfection function, it can permeate into every tiny corner with a proper concentration and pressure to achieve the disinfection function. In the present invention, a rotatable equipment box is used to perform full disinfection without any blind spot. In addition, ultraviolet disinfection lamps, air dehumidification equipment and ozone generation equipment are used collaboratively, so that the medical equipment can be disinfected quickly and sufficiently in a short time to save time.

To achieve the abovementioned object, the present invention provides a disinfection box. The disinfection box includes a disinfection box unit, ozone generation equipment and air dehumidification equipment. The disinfection box unit defines a holding space to provide an equipment box and at least an ultraviolet disinfection lamp. The ozone generation equipment is connected to the disinfection box unit and transmits the generated ozone to the disinfection box unit. The air dehumidification equipment is connected to the disinfection box unit and dries air inside the disinfection box unit.

According to an embodiment of the present invention, the ozone generation equipment includes an ozone generator which is connected to the holding space and outputs ozone, as well as an ozone reductor which is connected to the holding space and inputs ozone.

According to an embodiment of the present invention, the equipment box is driven to rotate by a motor.

According to an embodiment of the present invention, the air dehumidification equipment is connected to air filtration equipment and an air pump which is connected to the air filtration equipment; whereas, the air pump transmits the filtered air to the air dehumidification equipment.

A secondary object of the present invention is to provide a disinfection box wherein the ozone generator provides dry air inside the ozone generator to increase the lifetime of the ozone generator.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a planar view of a preferred embodiment according to the present invention.

FIG. 2 shows a schematic view of a usage state of an equipment box, according to the present invention.

FIG. 3 shows a schematic view of a usage state when the present invention performs drying, sterilization, deodorization and air cleaning.

FIG. 4 shows a schematic view of a usage state when the present invention reduces ozone.

### DETAILED DESCRIPTION OF THE PREFERRED EMBODIMENTS

The implementation of the present invention is hereinafter described by a certain specific embodiment, and those skilled in the art disclosed in this specification may readily understand other advantages and effects of the present invention by the contents disclosed in this specification.

The structure, proportion, and size depicted in the accompanying drawings in this specification are only provided for those skilled in the art to read and understand, along with the contents disclosed in the specification, and are not intended to limit the conditions under which the present invention may be implemented. Therefore, they do not have any technical meaning, and any modification to the structure, change in the proportion or adjustment of the size should still fall within the scope of technical contents disclosed by the present invention, without affecting the efficacy and the object that the present invention can reach. Meanwhile, the terms cited in this specification, such as “one”, “two”, and “upper” are also merely used to facilitate description and not intended to limit the range that the present invention may be practiced. The change or adjustment n their relative relationships is also considered to be within the scope of the implementation of the present invention, if there is no substantial change in the technology contents.

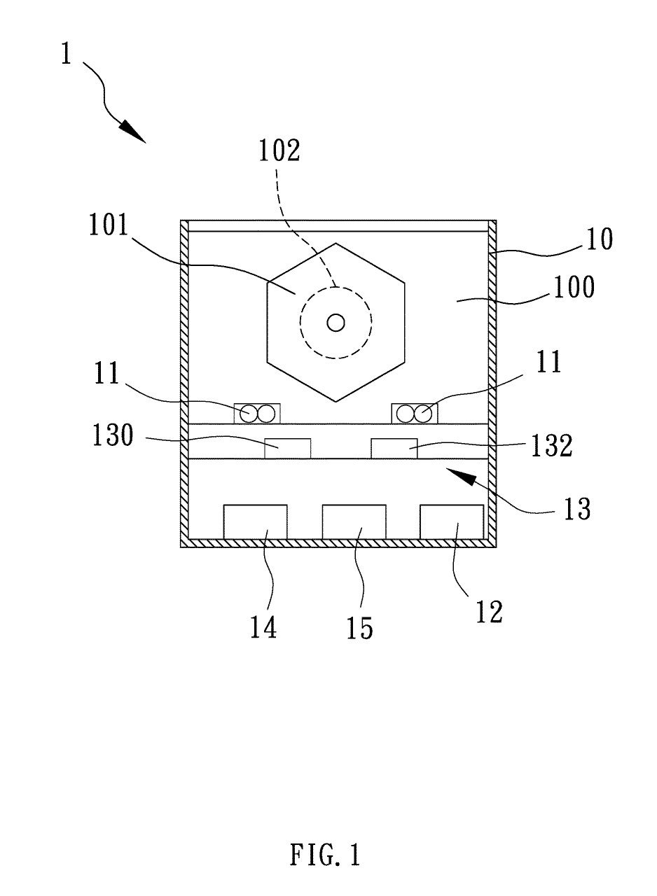

Referring to FIG. 1, it shows a planar view of a preferred embodiment according to the present invention. The present invention discloses a disinfection box 1 which comprises primarily a disinfection box unit 10, ultraviolet disinfection lamps 11, air dehumidification equipment 12 and ozone generation equipment 13. The disinfection box unit 10 defines a holding space 100 for emplacing an equipment box 101, and an interior of the holding space 100 is provided with the ultraviolet disinfection lamps 11.

In addition, the disinfection box unit 10 is connected to the air dehumidification equipment 12 and the ozone generation equipment 13, wherein the air dehumidification equipment 12 and the ozone generation equipment 13 are all connected to the disinfection box unit 10. The ozone generation equipment 13 includes an ozone generator 130 which is connected to the holding space 100 and outputs ozone, as well as an ozone reductor 132 which is connected to the holding space 100 and inputs ozone. The air dehumidification equipment 12 is connected to air filtration equipment 14 and an air pump 15 which is connected with the air filtration equipment 14; whereas, the air pump 15 transmits filtered air to the air dehumidification equipment 12.

The equipment box 101 inside the disinfection box unit 10 is used primarily to emplace medical equipment, and can be driven to rotate by a motor 102. As the ultraviolet disinfection lamps 11 are installed in the holding space 100, the medical equipment to be disinfected will be disinfected fully without any blind spot by the rotatable equipment box 101. In addition, by the collaborative use of the ultraviolet disinfection lamps 11, the air dehumidification equipment 12 and ozone generated by the ozone generation equipment 13, the medical equipment to be disinfected can be disinfected and sterilized quickly and sufficiently in a short time, thereby achieving the purposes of saving time as well as being sterilized and disinfected without any blind spot.

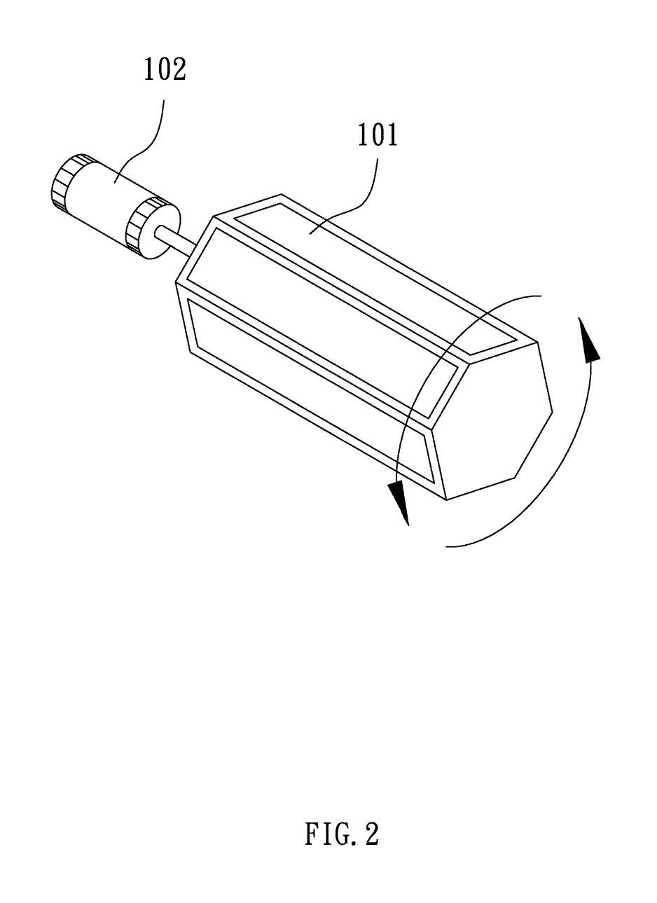

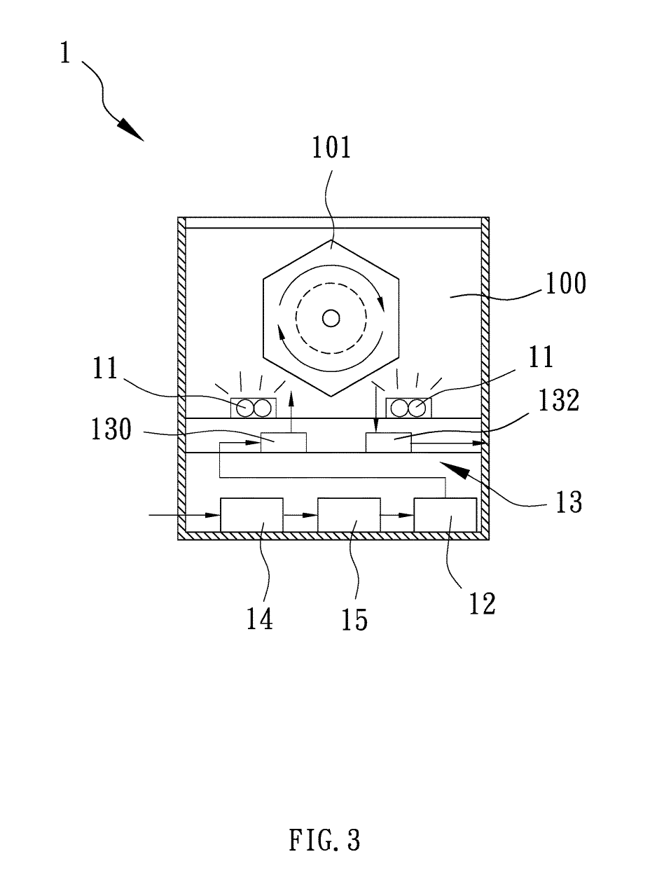

Referring to FIGS. 2 to 4 together, it shows a schematic view of a usage state of the equipment box, a schematic view of a usage state when the present invention performs drying, sterilizing and deodorizing, and air cleaning, and a schematic view of a usage state when the present invention reduces ozone. The arrows in the drawings point to the direction of transmission of air and ozone, and the direction of rotation of the equipment box 101. As shown in FIG. 3, upon performing disinfection to the medical equipment, the medical equipment are put into the equipment box 101, and then the air pump 15 is activated to withdraw air. At this time, air outside the disinfection box unit is filtered preliminarily by the air filtration equipment 14 to keep the inlet air clean and free of dusts. Next, extra water is removed from the filtered air through the air dehumidification equipment 12, allowing the dehumidified air to enter into the ozone generator 130 that the air entering into the holding space 100 will be dry and free of dusts. In addition, the ozone generator 130 will generate ozone and input ozone into the holding space 100. By the collaborative use of the air dehumidification equipment and the ozone generation equipment, the medical equipment can be dried and deodorized in a short time.

In a mean time, the ultraviolet disinfection lamps 11 installed at a periphery of the equipment box 101 will generate ultraviolet light to sterilize and disinfect the medical equipment simultaneously. As the equipment box 101 is driven to rotate by the motor 102, the medical equipment to be disinfected can be disinfected fully without any blind spot. Furthermore, by the collaborative use of the ultraviolet disinfection lamps 11, the air dehumidification equipment 12 and the ozone generated by the ozone generation equipment 13, the medical equipment to be disinfected can be sterilized and disinfected quickly and sufficiently in a short time, thereby achieving the purposes of saving time as well as being sterilized and disinfected without any blind spot.

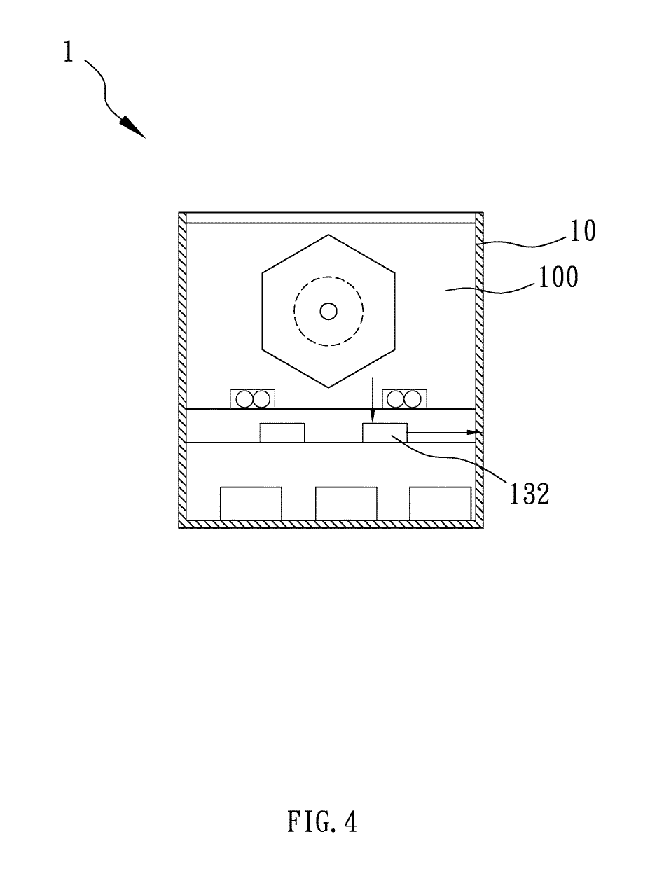

As shown in FIG. 4, after accomplishing sterilization with ozone, the reduction of ozone in the disinfection box unit 10 is required. Therefore, the ozone reductor 132 is used to filter ozone inside the disinfection box unit that the air with ozone is filtered and the air without ozone is discharged, thereby reducing the air with ozone to ordinary air.

In the present invention, the equipment box is designed to be rotatable, so that the medical equipment can be disinfected fully without any blind spot. In addition, by the collaborative use of the ultraviolet disinfection lamps 11, the air dehumidification equipment 12 and the ozone generation equipment 13, the medical equipment can be disinfected quickly and sufficiently in a short time, thereby saving time.

It is worth mentioning that when medical ozone is generated, it is usually under various concentrations, and the ratio of the quantity of ozone to the quantity of pure oxygen for medical use is called the percentage concentration, in a unit of micrograms per milliliter (□g/ml). Under the standard condition, one liter of oxygen weights by 1.4 g, where the standard condition usually means that temperature is 0° C. (or 273.15° K.) and pressure (or pressure strength) is 101.325 kPa (1 standard atmospheric pressure or 760 mmHg). Ozone at a concentration of 60 □g/ml is taken as an upper limit in ozone therapy, as the intravenous ozone injection of ozone at the concentration of more than 60 □g/ml may result in red cell infolding, which in turn can cause hemolysis. The existing experiments show that ozone at the concentration of more than 72 □g/ml can inhibit the growth of healthy cells, and if the ozone concentration is kept less than 60 □g/ml, it will cause no problem in the treatment such as ozone autologous blood transfusion.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A disinfection box comprising:
a disinfection box unit, which defines a holding space to provide a rotatable equipment box and ultraviolet disinfection lamps;
ozone generation equipment, which is connected to the disinfection box unit and transmits the generated ozone to the disinfection box unit; and
air dehumidification equipment, which is connected to the disinfection box unit and dries air inside the disinfection box unit.

2. The disinfection box according to claim 1, wherein the ozone generation equipment includes an ozone generator which is connected to the holding space and outputs ozone, as well as an ozone reductor which is connected to the holding space and inputs ozone.

3. The disinfection box according to claim 1, wherein the equipment box is driven to rotate by a motor.

4. The disinfection box according to claim 1, wherein the air dehumidification equipment is connected to air filtration equipment and an air pump which is connected to the air filtration equipment and transmits the filtered air to the air dehumidification equipment.
